(19) United States
(12) Patent Application Publication
(10) Pub. No.: US 2024/0426830 A1
(43) Pub. Date: Dec. 26, 2024

(54) CHRONIC WOUND HEALING BIOMARKER DIAGNOSTICS

(71) Applicant: THE TRUSTEES OF INDIANA UNIVERSITY, Bloomington, IN (US)

(72) Inventors: Chandan K. SEN, Indianapolis, IN (US); Sashwati ROY, Indianapolis, IN (US); Lava TIMSINA, Zionsville, IN (US); Amitava DAS, Indianapolis, IN (US)

(21) Appl. No.: 18/698,172

(22) PCT Filed: Oct. 20, 2022
(86) PCT No.: PCT/US2022/047248

§ 371 Date: Apr. 3, 2024

Related U.S. Application Data
(60) Provisional application No. 63/270,868, filed on Oct. 22, 2021.
(60) Provisional application No. 63/413,244, filed on Oct. 4, 2022.

(51) Int. Cl. G01N 33/68

(52) U.S. Cl. CPC G01N 33/6815; G01N 33/6848; G01N 2800/56

(57) ABSTRACT

Disclosed herein are compositions and methods for rapid identification of wounds that are refractory to standard wound care.

### CROSS REFERENCE TO RELATED APPLICATIONS

This application claims priority to U.S. Provisional Patent Application Nos. 63/270,868, filed on Oct. 22, 2021 and 63/413,244 filed on Oct. 4, 2022, the disclosures of which are expressly incorporated herein.

### BACKGROUND

The wound fluid that bathes wound tissue reflects the overall wound microenvironment and shapes the functional response of wound-related cells. Metabolic pathways supply the high amounts of energy supply required for effective wound healing. More particularly, in healing wounds, successful flow of metabolic pathways will leave a specific metabolite footprint detectable in the wound fluid. In non-healing wounds, arrest of certain components of the metabolic pathways will leave a unique metabolite footprint. Differences between these the biochemical footprints of healing vs non-healing wounds helps identify specific biomarkers of healing and these biomarkers can provide the basis of point of care diagnostic tests to provide an early identification of wounds in need of enhanced care.

The 2020 CDC national diabetes statistics report estimated that 34.2 million Americans, or 10.5% of the population had diabetes in 2018. It is estimated that over the course of their lifetime up to a third of these people with diabetes will develop a diabetic foot ulcer (DFU). The complex pathogenesis of diabetic foot ulcers (DFUs) often makes them refractory to standard of care (SoC). The five year mortality and direct costs of care for people with diabetic foot complications are comparable to cancer. Approximately 40-60% of nontraumatic lower limb amputations worldwide are caused by diabetic complications, and 80% of these amputations follow DFU. In patients with hard-to-heal DFUs, treatment with multiple therapies is often necessary to manage stubborn wounds. The current treatment algorithm for DFU uses a failure to improve (>50% wound area reduction) after four weeks of standard of care (SoC) therapy to make a clinical decision on changing the therapy. The lack of objective early indicators of wound healing outcomes handicaps DFU care strategy, as in many cases, such loss of time is a critical contribution to limb loss. Robust biomarkers that predict non-healing (i.e., refractory to SoC) would provide an objective basis for early adoption of alternate/aggressive treatment regimen to wound care providers. Biomarkers that predict non-healing (i.e., refractory to SoC) would provide an objective basis for rationally adopting alternate treatment regimen to wound care providers in a timely manner, including use of advanced therapies selected from debridement, negative pressure therapies, electrical stimulation, as well as compression therapy and surgical procedures to alleviate ischemia often associated with chronic wounds. In particular, sharp debridement removes nonviable tissue and slough along with bacterial biofilms that prolong the inflammatory response in the chronic wound.

The FDA-NIH Biomarker working group defines biomarker as a characteristic that is measured as an indicator of a normal biological process, a pathogenic process, or a response to an exposure or intervention, including a therapeutic intervention. Biomarkers are qualified with emphasis on benefit-risk relationships, analytical validation and grading the level of evidence.

The present disclosure is directed to the use of a primary and a secondary biomarker that would predict healing of chronic wounds. The biomarkers disclosed herein are measured from wound fluid samples collected from the wound patient and can be measured at the point of care. The diverse etiology of chronic wounds makes them refractory to SoC. Limited response to SoC often results in loss of time which could be a serious factor leading to amputation. Biomarkers that predict non healing (i.e., refractory to SoC) would provide an objective basis for adopting alternate/aggressive treatment regimen to wound care providers. Our premise is that non-healing (diabetic, ischemic and others) wounds suffer from metabolic impairments which in turn would be reflected in the metabolite profile in the wound fluid. The use of the wound fluid that bathes the entire tissue would be a closer representation of the composite view of the physiological/pathological state of the tissue. Therefore, the low molecular weight metabolites (LMWM) such as amino acids, carbohydrates, cofactors, vitamins, nucleotides, xenobiotics, peptides present in the bathing wound fluid, which are inherently more stable than proteins or genes, may serve as useful & reliable biomarkers of wound healing outcomes.

As disclosed herein applicant asserts that non-healing diabetic wounds suffer from metabolic impairments which in turn would be reflected by changes in metabolites contained in WF. Low molecular weight metabolites, inherently more stable than proteins or genes, will serve as robust biomarkers predicting DFU outcomes. One aspect of the present disclosure is directed to establishing a robust, reliable biomarker, or metabolite footprint, for early detection and prediction non-healing wounds.

### SUMMARY

The present disclosure is directed to enhancing the speed of diagnosing chronic wounds, particularly in diabetic patients, to allow for more rapid application of advanced therapeutic treatment to wounds in need of care that goes beyond standard of care procedures. Chronic wounds are heterogeneous with pockets of infection, inflammation and necrosis. This compromises the value of any one biopsy as a representation of the wound tissue. Punch biopsies therefore are often not a true and robust representation of the entire wound tissue environment, thus adding to the delay in the identification and treatment of chronic wounds. As an alternative, the collection of wound fluid (WF) that bathes the wound tissue provides a more accurate reflection of the overall wound tissue environment as the abundance of metabolites in this fluid is expected to be in equilibrium. Thus, compared to tissue biopsy, wound fluid is less invasive and is a better choice of sampling the wound microenvironment.

In a clinical setting numerous factors are simultaneously at play in determining wound healing outcomes, including those for diabetic wounds such as diabetic foot ulcers (DFU). The present disclosure is focused on one of the most downstream effectors of healing-wound tissue metabolism. Regardless of upstream factors, if an intervention is effective it must have a favorable impact on wound tissue metabolism. Tissue repair depends on oxidative metabolic processes at the site of injury, and non-healing wounds are characterized by metabolic impairments. As the stalled or blunted metabolic engine resumes activity, such favorable change will be reflected in the abundance of specific metabolic byproducts contained in the WF.

In accordance with the present invention specific biomarkers detected in wound fluids are utilized to identify wounds that will be refractory to standard care and will required advanced and more aggressive treatment protocols. In one embodiment the wound fluid diagnostic markers will be metabolites including for example, thiols bearing metabolites (of either low or high molecular weight).

In accordance with one embodiment of the present disclosure, applicant has discovered that low (<3.43 cut-off; see Table 1) cysteine to cystine ratio in wound fluid (WF) is a reliable biomarker to predict DFU non-healing (FIG. 4B). In one embodiment wound fluid will be stabilized and assayed for Cys and CysS using techniques known to the skilled practitioner, including for example, a microtiter plate assay (common assay platform in clinical testing) and Cys/CysS ratios will be calculated. This candidate biomarker was identified using an unbiased metabolomics (discovery phase) analysis of 161 chronic wound fluids (n=76 diabetic ulcers) and preliminary verification was performed using the proposed assay in n=24 DFU wound fluids.

In one embodiment a diagnostic tool is provided that predicts the healing outcomes of chronic wounds. Stable metabolites of the following categories: amino acids, carbohydrates, cofactors, vitamins, nucleotides, xenobiotics and peptides, that can be detected at point of care from wound fluid, can serve as biomarkers of chronic wound healing. Specific examples include:
1) amino acids or amino acid analogs, including for example 5-hydroxyproline, c-glycosyltryptophan, cysteinylglycine, guanidinoacetate, kynurenine, N(1)-acetylspermine, N1,N12-diacetylspermine, and taurine;
2) carbohydrates, cofactors, vitamins, nucleotides, xenobiotics: 3-phosphoglycerate, fructose 1,6-diphosphate/glucose 1,6-diphosphate/myo-inositol diphosphates, glucose, maltose, phosphoenolpyruvate, ascorbate, 5-methyluridine, cytidine 3′-monophosphate, cytidine 5′-monophosphate, guanosine 3′-monophosphate, guanosine 5′-monophosphate, inosine, uridine 2′-monophosphate, ergothioneine, levulinate, methyl-4-hydroxybenzoate, 3-phosphoglycerate; and
3) peptides, including for example: glycylglutamate, or isoleucyltyrosine. In addition any combination of the markers identified in 1) to 4) can be used (optionally as part of a panel) to distinguish between healing and non-healing wounds and identify wounds in need of advanced wound healing therapy beyond standard care.

In accordance with one embodiment the metabolites detected in would fluids to identify non-healing wounds include one or more compounds selected from the group consisting of glycylglutamate, cysteine, pro-hydroxy-pro, 5-hydroxylysine, prolylserine, cystine, taurine, cysteine s-sulfate, oleoyl ethanolamide, pregnenediol disulfate (C₂₁H₃₄O₈S₂), cytidine 5′-diphosphocholine, 1-(1-enyl-oleoyl)-GPE (P-18:1), 3-methoxycatechol sulfate (1), cytidine 3′-monophosphate (3′-CMP), phosphoethanolamine, gamma-glutamylphenylalanine, choline phosphate, methionylvaline, O-sulfo-L-tyrosine, guanosine 5′-monophosphate (5′-GMP), N6-carbamoylthreonyladenosine, cytidine, 3-hydroxybutyrylcarnitine (1), valyltyrosine, and methionylalanine. More particularly, identification of elevated levels (relative to levels detected in healing wounds) of glycylglutamate, pro-hydroxy-pro, 5-hydroxylysine, prolylserine, cystine, cysteine s-sulfate, oleoyl ethanolamide, pregnenediol disulfate (C21H34O8S2), 1-(1-enyl-oleoyl)-GPE (P-18:1)*, 3-methoxycatechol sulfate (1), gamma-glutamylphenylalanine, O-sulfo-L-tyrosine, N6-carbamoylthreonyladenosine, and/or cytidine, or decreased levels (relative to levels detected in healing wounds) of cysteine, taurine, cytidine 5′-diphosphocholine, cytidine 3′-monophosphate (3′-CMP), phosphoethanolamine, choline phosphate, methionylvaline, guanosine 5′-monophosphate (5′-GMP), 3-hydroxybutyrylcarnitine (1), valyltyrosine, and/or methionylalanine are diagnostic of non-healing wounds.

The levels or ratios of these molecules are also of diagnostic value and the respective markers can be elevated or decreased in non-healing wounds compared to healing wounds. These tools when used in the context of chronic wounds can aid in wound management planning and prevention of serious outcomes such as amputation. In one embodiment a method of rapidly identifying a patient as having a chronic wound is conducted by obtaining a wound fluid sample from a patient's wound, and measuring in said wound fluid sample the relative concentration of any of the wound tissue markers disclosed herein. In one embodiment wound fluids can be collected at point-of-care easily using occlusive dressing or vac sponges from negative pressure wound therapy (NPWT) and the fluid itself can be analyzed for the stable metabolite biomarkers listed herein.

In accordance with one embodiment a method of treating a chronic wound in a patient, including a diabetic patient with DFU is provided, wherein the first step is the rapid diagnosis of wounds that are refractive to standard care. In one embodiment a method of rapidly identifying a patient as having a chronic wound is conducted by obtaining a wound fluid sample from a patient's wound, and measuring in said wound fluid sample the relative concentration of one or more metabolites, wherein the detection of a metabolite profile associated with non-healing wounds identifies those wounds as chronic wounds in need of advanced wound healing therapy beyond the stand of care for wounds. In one embodiment the relative concentration of cysteine and cystine is measured in a wound fluid recovered for the patient, wherein a lower cysteine to cystine ratio (e.g., having ratio less than 3.4, 3.0, 2.5, 2.0 or 1.5) identifies a chronic wound. In one embodiment, patients identified as having a chronic wound will then have their wounds treated with advanced wound healing therapy. In one embodiment the wound to be treated is a DFU. In one embodiment the advanced therapy includes one or more therapies selected from the group consisting of wound debridement, negative pressure therapies, electrical stimulation, compression therapy and surgical procedures to alleviate ischemia.

In one embodiment the method of treating chronic wounds in a subject comprises
a) receiving an identification of a subject as having a chronic wound, wherein the chronic wound has been identified by an altered metabolite profile relative to the standard metabolite concentrations in healing wound fluids. In one embodiment the altered metabolite profile is the detection of a low cysteine to cystine ratio (e.g., having a ratio less than 3.4, 3.0, 2.5, 2.0 or 1.5) in would fluid recovered from the subject's wound; and
b) administering advanced wound healing therapy to the subject identified as having a chronic wound.

### BRIEF DESCRIPTION OF THE DRAWINGS

FIG. 1 is a Volcano plot representing low molecular weight metabolites that were significantly dysregulated between healing (H) and non-healing (NH) chronic wounds. Statistical testing for the two groups were performed using Student's t-test with unequal variance and data were represented as volcano plot. In NH wounds, Cys was significantly down-regulated (log2 fold change (FC)=1.35, Student's t-test p=5.02 E-06) and CysS was significantly upregulated (log2 FC=−1.58, Student's t-test p=4.01E-06). Select metabolites showing significant dysregulation also included: GG:glycylglutamate, 5HL:5-hydroxylysine, PHP:pro-hydroxy-proline, C:cytidine, 2HH:2-hydroxyhippu, BH:beta-hydroxyls, N:naproxen, G:guanine, E:ergothioneine and GS:guanosine. For brevity and space, select metabolites are labelled.

FIG. 2: Partial Least Squares Discriminant Analysis (PLSDA) analysis showed significant separation of non-healing samples from healing chronic wound fluid samples. PLSDA analysis investigated the covariance between the H and NH wound fluid samples with a model involving 578 metabolites. The first two components were represented as 2D scores plot with highlighted 95% confidence region. FIG. 2 provides Variable Importance in Projection (VIP) score (a measure of a variable's importance) computed in PLSDA, indicate Cys (VIP=3.28, rank=1st) and CysS (VIP=2.78, rank=8th) scored among the top 15 out of 578 metabolites for component 1. Consistently, Cys was downregulated and CysS was upregulated in NH wounds.

FIGS. 3A-3C represent Box plot representation of Cysteine (Cys; FIG. 3A) and cystine (CysS; FIG. 3B) in healing (H) non-healing diabetic wounds (NH). FIGS. 3A and 3B present normalized concentrations of Cys and CysS in healing (H) non-healing diabetic wounds. FIG. 3C presents the ratio of Cys to CysS in healing (H) versus non-healing (NH) diabetic wounds (n=76). Based on the median regression with bootstrapped standard error obtained after 500 iterations, among all diabetic ulcers, the Cys/CysS was significantly downregulated (˜7-fold) in NH wounds compared to H wounds lower as indicated by β-coefficient.

FIG. 4: Kit-based analysis of cysteine/cystine ratio in diabetic foot ulcer (DFU) samples. Human DFU wound fluid samples (n=24) were analyzed using cysteine assay kit (fluorimetric) (Abcam, MA, ab211099). Healing (>65% closed after 4 wks) (n=9) and non-healing (<20% closed after 4 wks) (n=15) were included in the analysis. Non-healing wounds showed significantly lower cysteine to cystine ratio compared to healing wounds. Data represents mean SEM. *p<0.05 compared to healing.

FIG. 5: Sensitivity vs specificity analysis for Cys/CysS in diabetic ulcers. The receiver operator characteristics (ROC) analysis of these biomarkers with wound healing outcome among all diabetic ulcers revealed that the area under the curve (AUC) for Cys/CysS was 0.81. Analysis included n=76 diabetic ulcer samples.

FIG. 6A-6C: presents data showing bivariate (FIG. 6A) and multivariable (FIG. 6B) analysis indicated two common metabolites (5-hydroxylysine and cystine) that are upregulated among non-healers. FIG. 6C represents only those amino acids that are significant in both bivariate and multivariable analysis with individual level of quantitation of the metabolites stratified by the wound healing status, wherein for each lane the left column represents healing and the right column represent non-healing would fluids and the metabolites measured are 3-phosphoglycerate (lane 1), fructose 1,6-diphosphate/glucose 1,6-diphosphate/myo-inositol diphosphates (lane 2), glucose (lane 3), maltose (lane 4), phosphoenolpyruvate (PEP) (lane 5), ascorbate (vitamin C) (lane 6), 5-methyluridine (ribothymidine) (lane 7), cytidine 3′-monophosphate (3′-CMP) (lane 8), cytidine 5′-monophosphate (5′-CMP) (lane 9), guanosine 3′-monophosphate (3′-GMP) (lane 10), guanosine 5′-monophosphate (5′-GMP) (lane 11), inosine (lane 12), uridine 2′-monophosphate (2′-UMP)* (lane 13), ergothioneine (lane 14), levulinate (4-oxovalerate) (lane 15) and methyl-4-hydroxybenzoate (lane 16).

FIGS. 7A-7C: presents the results for measured peptides from healing and non-healing wounds. Both, univariate (FIG. 7A) and multivariable (FIG. 7B) analysis indicated that only glycylglutamate was upregulated among non-healers and isoleucyltyrosine was downregulated among non-healers compared to healers. FIG. 7C represents only those peptides that are significant in both bivariate and multivariable analysis with individual level of quantitation of the metabolites stratified by the wound healing status.

FIG. 8: Volcano plot representing low molecular weight metabolites that were significantly dysregulated between healing (H) and non-healing (NH) chronic wounds. Statistical testing for the two groups were performed using Student's t-test with unequal variance and data were represented as volcano plot.

FIG. 9: Partial least squares-discriminant analysis (PLS-DA) based on metabolomics data between healers and non-healers was conducted and the VIP scores of high ranking metabolite is presented.

### DETAILED DESCRIPTION

### Definitions

In describing and claiming the invention, the following terminology will be used in accordance with the definitions set forth below.

The term “about” as used herein means greater or lesser than the value or range of values stated by 10 percent but is not intended to limit any value or range of values to only this broader definition. Each value or range of values preceded by the term “about” is also intended to encompass the embodiment of the stated absolute value or range of values.

As used herein, the term “purified” and like terms relate to the isolation of a molecule or compound in a form that is substantially free of contaminants normally associated with the molecule or compound in a native or natural environment. As used herein, the term “purified” does not require absolute purity; rather, it is intended as a relative definition. The term “purified polypeptide” is used herein to describe a polypeptide which has been separated from other compounds including, but not limited to nucleic acid molecules, lipids and carbohydrates.

The term “isolated” requires that the referenced material be removed from its original environment (e.g., the natural environment if it is naturally occurring). For example, a naturally-occurring polynucleotide present in a living animal is not isolated, but the same polynucleotide, separated from some or all of the coexisting materials in the natural system, is isolated.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, the term “treating” includes prophylaxis of the specific disorder or condition, or alleviation of the symptoms associated with a specific disorder or condition and/or preventing or eliminating said symptoms.

As used herein an “effective” amount or a “therapeutically effective amount” of a drug refers to a nontoxic but enough of the drug to provide the desired effect. The amount that is “effective” will vary from subject to subject or even within a subject overtime, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

As used herein the term “patient” without further designation is intended to encompass any warm blooded vertebrate domesticated animal (including for example, but not limited to livestock, horses, cats, dogs and other pets) and humans receiving a therapeutic treatment in the presence or absence of physician oversight.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “inhibit” refers to a decrease in an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

As used herein the term “metabolome” defines the complete set of small-molecule chemicals found within a biological sample. The biological sample can be a cell, a cellular organelle, an organ, a tissue, a tissue extract, a bodily fluid or an entire organism.

Chronic wounds are defined herein as any wound that fails to proceed through the normal phases of wound healing in an orderly and timely manner.

### EMBODIMENTS

Reliable sampling of the wound microenvironment is necessary as a foundation to biomarker exploration. Several reported studies addressing biomarker analysis have relied on wound fluid as the source material because of its value as a representative sample of the internal wound microenvironment. Wound tissue biopsy or debrided tissue, being point measurements, are at risk of sampling error because it may come from a part of the heterogeneous wound that is not a true representation of the wound tissue. Wound tissue heterogeneity is well documented. Moreover, tissue biopsies are invasive.

In accordance with one embodiment wound fluid (WF) is used as the source material for assessing the wound microenvironment. The WF bathes large parts of the wound tissue and solutes in such fluid equilibrate. Furthermore, WF presents a composition that is stable in samples collected several times from the same wound.

Repair is a physiological response to healing. For successful repair to be achieved, numerous physiological factors work in co-ordination culminating in energy-producing tissue metabolism which is necessary to produce new tissue mass. Repair is derailed when barriers such as infection or chronic inflammation are insurmountable. No matter where these barriers are, the end result is that the metabolism required for tissue repair is blunted or stalled. Metabolomics is the systemic study of all metabolites and their concentration as affected by pathological and physiological factors. Metabolomics is a powerful approach to study the wound microenvironment as the balance of specific metabolites can provide direct insight into the process of wound healing. Therefore, early perturbations in the “normal” metabolome may be indicative of disease development. Such alterations in the metabolome footprint can serve as an early diagnostic indicator of a defective healing response.

In accordance with one aspect of the present disclosure would fluids from patients, particularly diabetic patients, are analyzed using analytical tools known to the skill practitioner (e.g. mass spectrometry or kit-based assays) to identify perturbed metabolome profiles of the wound fluid wherein the altered metabolome footprint, or specific components thereof, can be used to categorize the wound as a normal wound or a chronic wound. Rapid identification of chronic wounds allows such wounds to be treated at an early stage with advanced treatment therapies to speed wound repair

Metabolites are small molecule intermediates and end-products of biochemical reactions in tissue. Metabolomics may be viewed as a downstream footprint that accounts for functional interaction between numerous upstream modifiers of wound healing. Compared to traditional protein and gene biomarkers, low molecular weight metabolites (LMWM) have been reported to be more suitable as biomarkers because of their inherent stability and robustness.

In accordance with one embodiment of the present disclosure, wound fluid will be collected by standard techniques, including, for example, negative pressure wound therapy (NPWT) dressings or filter disks, and stabilized in a suitable buffer such as phosphate buffered saline (PBS) that optionally may include up to about 4% monochloro-acetic acid (MCA). In one embodiment the stabilized WFs will be flash frozen and stored in liquid nitrogen (liq. N₂) until analysis of the wound fluid metabolome.

In accordance with one embodiment of the present disclosure a highly rigorous and demanding two-step approach is used to identify candidate biomarkers of non-healing wounds. Wound fluid was collected from 161 different chronic wound patients, including 77 patients having diabetic ulcers, and 578 metabolites were analyzed using multivariable median regression for their association with non-healing (<20% closure over 4 weeks); compared to healing wounds (>65% closure over the same time period). Adjustments were made for the confounding effect of age, gender and diabetes status.

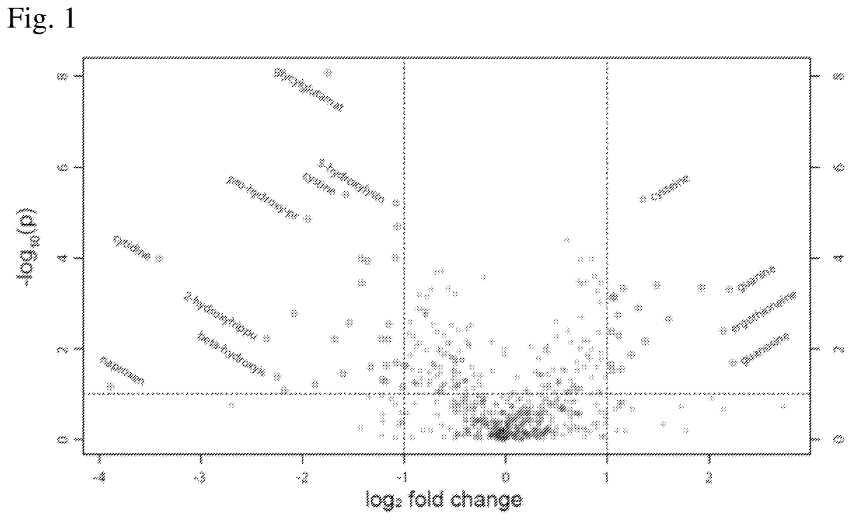

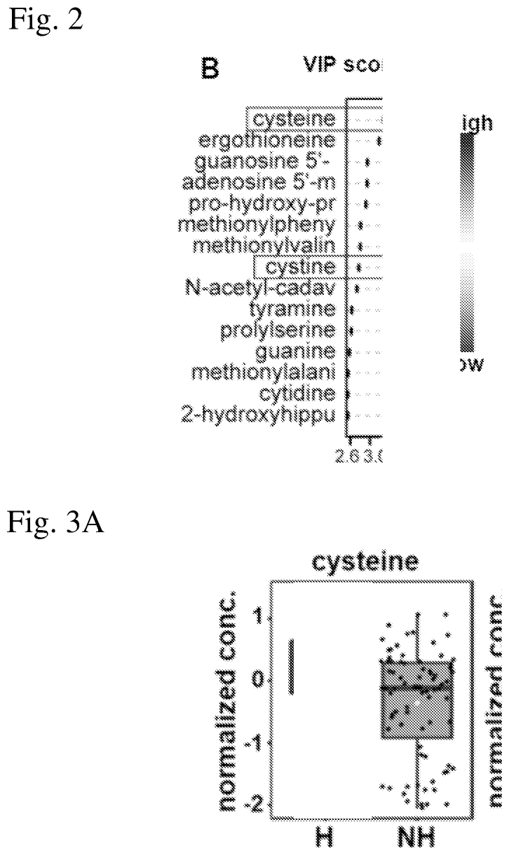

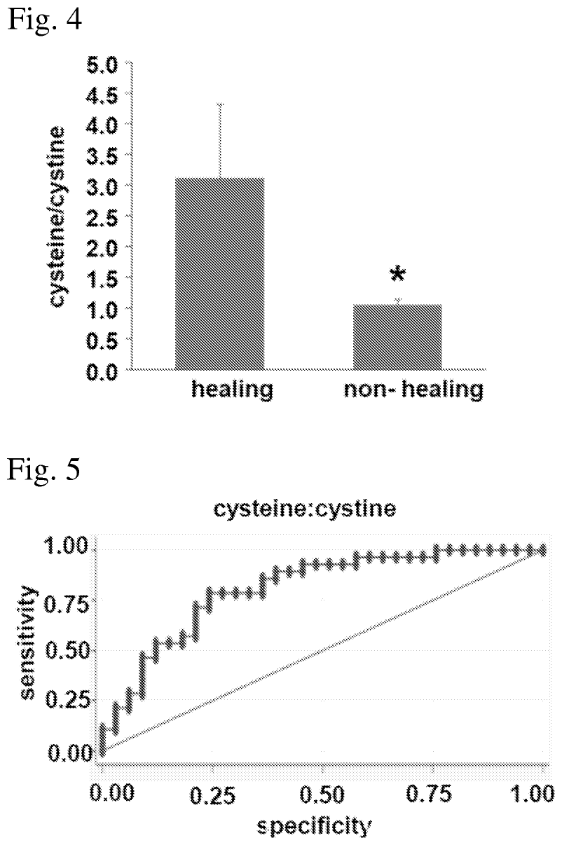

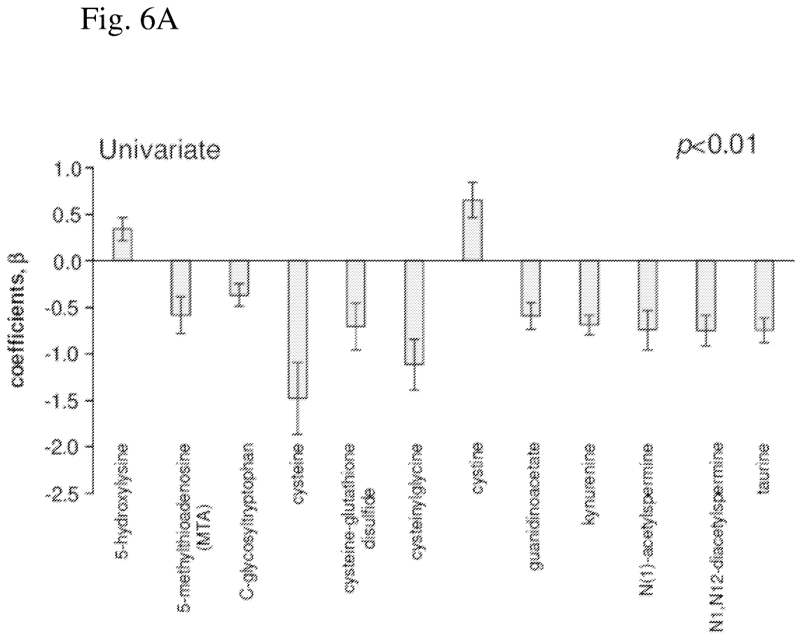

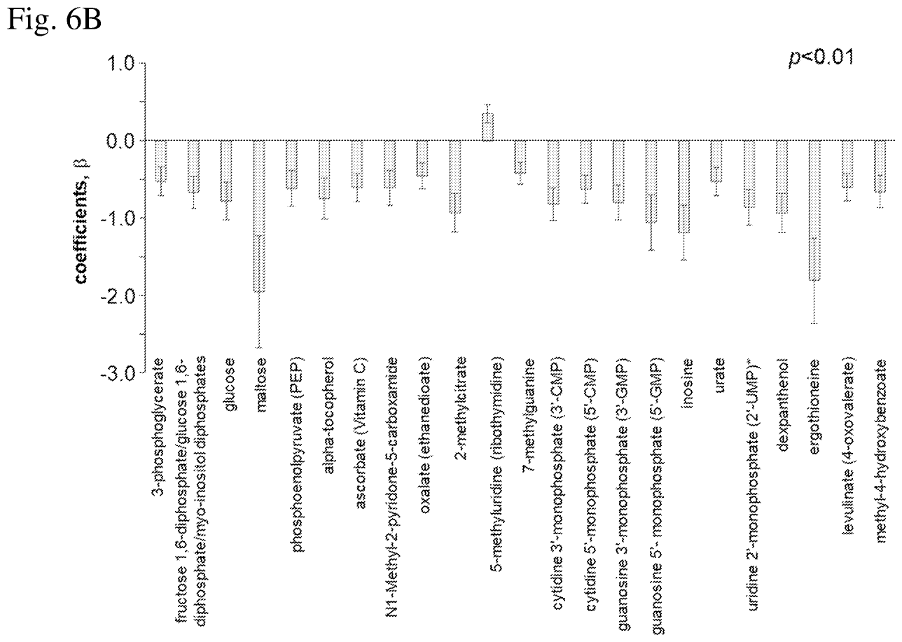

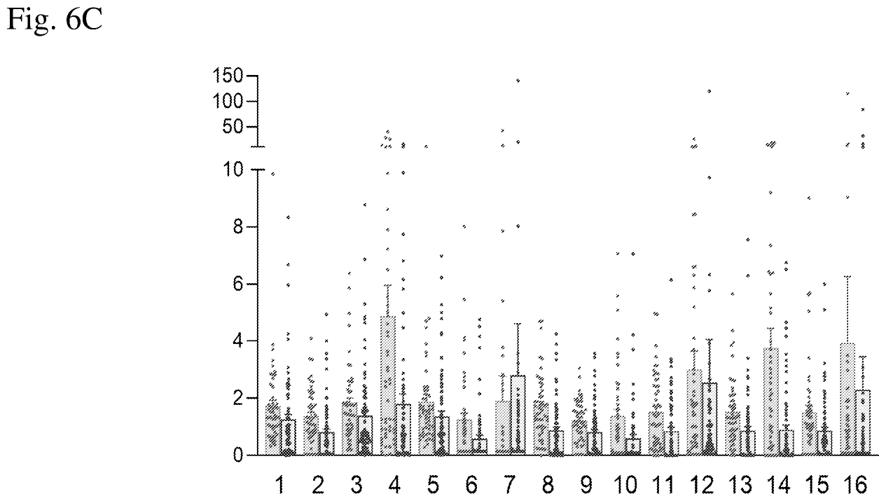

One of the analyzed metabolites, cysteine (coeff=1.52,*p<0.01) was found to be highly diminished and cystine (coeff=0.65,*p<0.01) was found to be highly increased in non-healing wound fluids compared to healing wound fluids. In accordance with one embodiment a robust ultrahigh performance liquid chromatography-tandem mass spectroscopy platform, combined with blinded bioinformatics analysis, was performed by an independent facility to detect and quantify wound fluid metabolites. A prospective validation pilot where N=24 DFU patients were enrolled was conducted, and one candidate biomarker that emerged from Step 1 (cysteine/cystine (Cys/CysS)) was tested using a point-of-care assay system. Exclusion criteria were minimized such that the study could include >90% of all DFU patients who presented at our wound clinic. Low Cys/CysS ratio was identified as the biomarker from Step 1 (FIGS. 1-3C). This was successfully validated in DFU patient WF in Step 2 (FIG. 4).

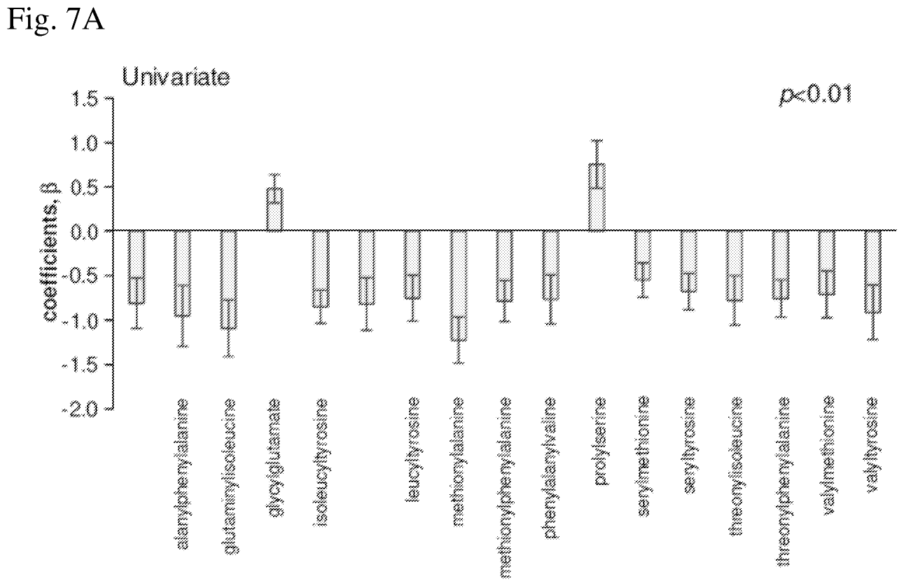

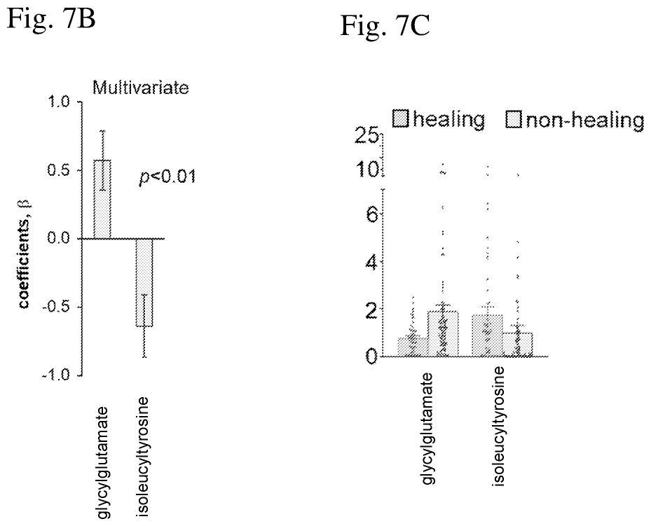

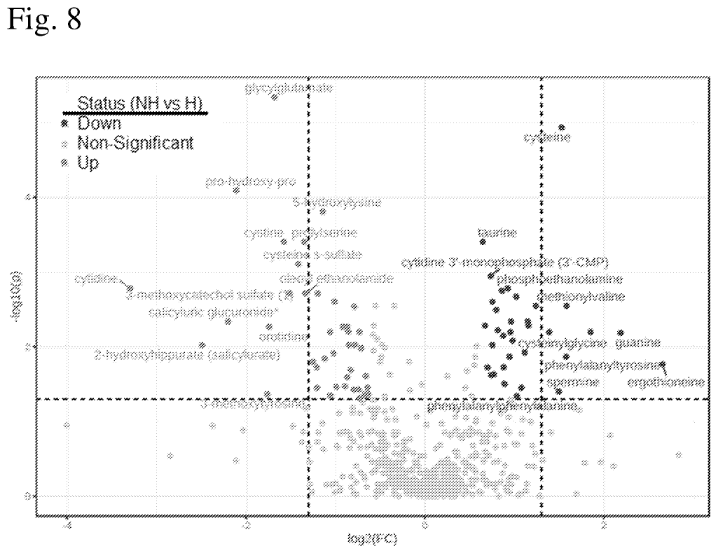

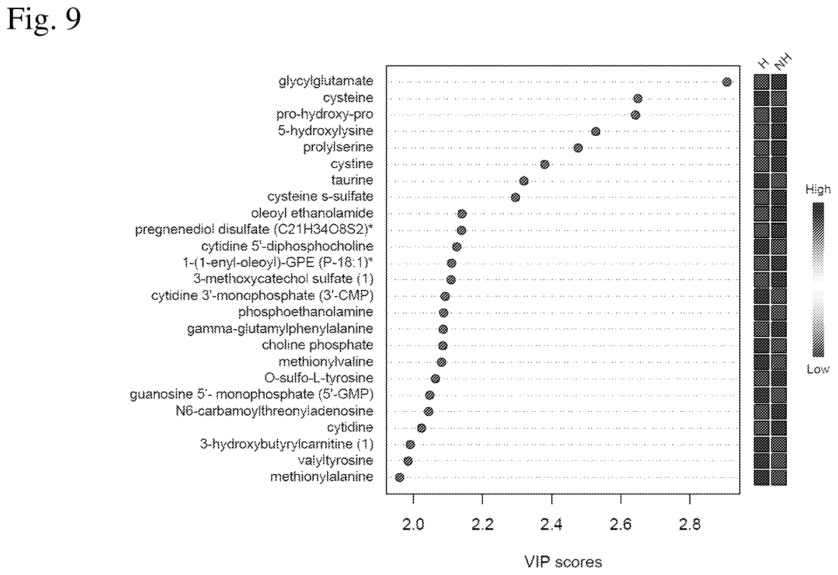

In one embodiment the WF will be collected using NPWT dressings or filter disks and stabilized in 1× phosphate buffered saline (PBS) with 4% monochloro-acetic acid (MCA). Stabilized WFs can be flash frozen and stored in liquid nitrogen (liq. N₂) for later analysis. In one embodiment the stabilized WF solution will be assayed for any of the components identified in FIGS. 1, 2, 7B, 8 or 9 as having altered concentrations in healing vs non-healing wounds.

In accordance with one embodiment the metabolites detected in would fluids to identify non-healing wounds include one or more compounds selected from the group consisting of glycylglutamate, cysteine, pro-hydroxy-pro, 5-hydroxylysine, prolylserine, cystine, taurine, cysteine s-sulfate, oleoyl ethanolamide, pregnenediol disulfate (C₂₁H₃₄O₈S₂), cytidine 5′-diphosphocholine, 1-(1-enyl-oleoyl)-GPE (P-18:1)*, 3-methoxycatechol sulfate (1), cytidine 3′-monophosphate (3′-CMP), phosphoethanolamine, gamma-glutamylphenylalanine, choline phosphate, methionylvaline, O-sulfo-L-tyrosine, guanosine 5′-monophosphate (5′-GMP), N6-carbamoylthreonyladenosine, cytidine, 3-hydroxybutyrylcarnitine (1), valyltyrosine, and methionylalanine.

In one embodiment the metabolites detected in would fluids to identify non-healing wounds include one or more compounds selected from the group consisting of glycylglutamate, cysteine, 5-hydroxylysine, pro-hydroxy-proline, prolylserine, cystine, taurine, cysteine s-sulfate, 2-hydroxyhippu, beta-hydroxyls, naproxen, guanine, ergothioneine and guanosine.

In one embodiment the metabolites detected in would fluids to identify non-healing wounds include one or more compounds selected from the group consisting of glycylglutamate, cysteine, 5-hydroxylysine, pro-hydroxy-proline, prolylserine, cystine, taurine, and cysteine s-sulfate.

In one embodiment the metabolites detected in would fluids to identify non-healing wounds include one or more compounds selected from the group consisting of glycylglutamate, 5-hydroxylysine, pro-hydroxy-proline, 2-hydroxyhippu, beta-hydroxyls, naproxen, cytidine, guanine, ergothioneine and guanosine.

In one embodiment the metabolites detected in would fluids to identify non-healing wounds include one or more compounds selected from the group consisting of glycylglutamate, cysteine, cystine and isoleucyltyrosine.

In one embodiment the ratio of the detected concentration of two metabolites detected in the wound fluid provides a diagnostic indicator of a non-healing wound, wherein ratio is between a first metabolite selected from cysteine, taurine, cytidine 5′-diphosphocholine, cytidine 3′-monophosphate (3′-CMP), phosphoethanolamine, choline phosphate, methionylvaline, guanosine 5′-monophosphate (5′-GMP), 3-hydroxybutyrylcarnitine (1), valyltyrosine, and methionylalanine and a second metabolite selected from the group consisting of glycylglutamate, pro-hydroxy-pro, 5-hydroxylysine, prolylserine, cystine, cysteine s-sulfate, oleoyl ethanolamide, pregnenediol disulfate (C21H34O8S2), 1-(1-enyl-oleoyl)-GPE (P-18:1), 3-methoxycatechol sulfate (1), gamma-glutamylphenylalanine, O-sulfo-L-tyrosine, N6-carbamoylthreonyladenosine, and cytidine.

In one embodiment the ratio of the detected concentration of two metabolites detected in the wound fluid provides a diagnostic indicator of a non-healing wound, wherein ratio is between a first metabolite selected from cytidine, guanine, ergothioneine and guanosine and a second metabolite selected from the group consisting of glycylglutamate, 5-hydroxylysine, pro-hydroxy-proline, 2-hydroxyhippu, beta-hydroxyls, and naproxen.

In one embodiment a non-healing wound is identified by the detection of elevated levels (relative to levels detected in healing wounds) of one or more metabolites selected from glycylglutamate, pro-hydroxy-pro, 5-hydroxylysine, prolylserine, cystine, cysteine s-sulfate, oleoyl ethanolamide, pregnenediol disulfate (C21H34O8S2), 1-(1-enyl-oleoyl)-GPE (P-18:1), 3-methoxycatechol sulfate (1), gamma-glutamylphenylalanine, O-sulfo-L-tyrosine, N6-carbamoylthreonyladenosine, and/or cytidine.

In one embodiment a non-healing wound is identified by the detection of decreased levels (relative to levels detected in healing wounds) of cysteine, taurine, cytidine 5′-diphosphocholine, cytidine 3′-monophosphate (3′-CMP), phosphoethanolamine, choline phosphate, methionylvaline, guanosine 5′-monophosphate (5′-GMP), 3-hydroxybutyrylcarnitine (1), valyltyrosine, and methionylalanine.

In one embodiment a non-healing wound is identified by the detection of elevated levels (relative to levels detected in healing wounds) of one or more metabolites selected from glycylglutamate, 5-hydroxylysine, pro-hydroxy-proline, 2-hydroxyhippu, beta-hydroxyls, and naproxen.

In one embodiment a non-healing wound is identified by the detection of an expression profile of metabolites wherein a specific altered concentration of a plurality of metabolites selected from the group consisting of glycylglutamate, cysteine, pro-hydroxy-pro, 5-hydroxylysine, prolylserine, cystine, taurine, cysteine s-sulfate, oleoyl ethanolamide, pregnenediol disulfate (C21H34O8S2), cytidine 5′-diphosphocholine, 1-(1-enyl-oleoyl)-GPE (P-18:1)*, 3-methoxycatechol sulfate (1), cytidine 3′-monophosphate (3′-CMP), phosphoethanolamine, gamma-glutamylphenylalanine, choline phosphate, methionylvaline, O-sulfo-L-tyrosine, guanosine 5′-monophosphate (5′-GMP), N6-carbamoylthreonyladenosine, cytidine, 3-hydroxybutyrylcarnitine (1), valyltyrosine, naproxen 2-hydroxyhippu, guanine, ergothioneine and methionylalanine is detected wherein the altered concentration profile is associated with non-healing wounds.

In one embodiment a non-healing wound is detected by measuring the concentration of cysteine (Cys) and cystine (CysS). In one embodiment the stabilized WF solution will be assayed for Cys and CysS using a microtiter plate assay (common assay platform in clinical testing) and the Cys/CysS ratio will be calculated. This candidate biomarker was identified using an unbiased metabolomics (discovery phase) analysis of 161 chronic wound fluids (n=76 diabetic ulcers) and preliminary verification was performed using the proposed assay in n=24 DFU wound fluids. The cut off based on Youden Index for healing rate was ≥3.43 which indicates that Cys/CysS<3.43 will serve as an indicator of non-healing trajectory.

WF collection via filter disc and/or NPWT wound dressings were selected as methods of choice. Steps must be taken to ensure cysteine stability in the collected samples. Autoxidation can cause thiol-disulfide exchange during sample preparation and storage. In addition, proteins with peptidase enzymatic properties may pose threat of artefacts. The combined acidifying and denaturing effects of monochloroacetic acid (MCA) manages both threats by virtue of its weak acid properties. The addition of MCA (4% W/V) results in protein precipitation, whereas low-molecular-mass thiols (Cys and CysS) stay stable.

WF from NPWT dressing or filter disks will be aliquoted and stored locally in weak acid under liquid nitrogen (displaces oxygen) and will be shipped to a Biomarker Analysis Unit (BAU) on dry ice in batches of 5 samples. The robustness of the assay depends upon how they are collected, stored and transported and therefore a standard operating protocol will be used for these steps. Working standards provided with the commercial kit will be used for calibration. In one embodiment the assay is conducted in a 96-well microtiter plate format, and quality control (QC) replicates will be included in each plate to measure accuracy for each individual sample batch that is run.

Assay selectivity/matrix interference test: In this test, 3-5 biological samples will be spiked with known amounts of Cys and CysS standard material and recovery of the added material will be evaluated. Specificity of detection of Cys and CysS will be evaluated using HPLC electrochemical detection. The most important measure of assay sensitivity in quantitative bioanalysis is the LLOQ; the lowest concentration of analyte that can be measured with an acceptable level of bias, precision and total error. LOD is the lowest amount of analyte that can be statistically distinguished from zero, but it cannot be quantified with certainty. Evaluation and definition of the LLOQ will be performed during assay validation to confirm suitability of the assay for the intended application.

The stability of candidate biomarker will be assessed using actual samples containing endogenous material. The variables that affect stability include sample collection, storage (at site or during transit) shipping and storage at the laboratory. To recapitulate storage and shipping conditions, at the BAU, samples at the site of recovery will be measured upon collection and aliquots will be frozen in liquid nitrogen and placed in dry ice for a 24-48 h period (based on the fact that biohazard sample shipping is typically overnight) and then assayed. Initial accuracy and precision acceptance criteria for sample analysis will be set using spiked buffer QC samples. Native or spiked MCs (MC pools) are preferred for evaluation of precision and relative accuracy during validation. Once the analyte concentration levels in MC pools have been established (after multiple runs involving multiple reagent lots), they can be used to set both accuracy and precision of quantification in routine testing, as well as facilitating trend analysis and monitoring of lot-to-lot consistency in assay performance.

In accordance with one embodiment a kit is provided for recovering and analyzing would fluids from patients. In one embodiment the kit comprises components for recovering wound fluids, including for example NPWT dressing or filter disks as well as reagents for stabilization and analysis of the wound fluid sample. In one embodiment the kit comprises a buffered solution comprising monochloroacetic acid (MCA). In one embodiment the MCA solution is formulated in a phosphate buffer such as 1×PBS and the final concentration of MCA is 4% W/V when mixed with the WF sample.

Initial ruggedness of a kit-based assay will be documented primarily with the accuracy and precision data generated during the validation exercise on different days and using multiple lots of kits or reagents. Samples collected at other DFC CRU sites will be tested for comparison and back-end validated using HPLC electrochemical detection. In one embodiment samples (NPWT dressing/disk filter) once collected can be processed and analyzed immediately by be transferring the samples to a 50 ml Falcon tube, washing with 1× phosphate buffer saline (PBS) and centrifuging at 300 RCF for 5 mins. A standard refrigerated table-top centrifuge used for plasma/serum separation is adequate to process samples. 100 ul of the 40% monochloroacetic acid (MCA) will be added to 900 ul of the supernatant (i.e., wound fluid) so that the final concentration of the monochloroacetic acid is 4%, mixed thoroughly, snap frozen in liquid nitrogen and stored in liquid nitrogen. This stabilized solution, optionally containing an internal standard, (10 μl volume used for assay per reaction) will be assayed for Cys and CysS. Aliquots (1 ml) of the processed fluid will be frozen in liquid nitrogen and stored at −80° C. until shipment to the BAU unit. In one embodiment the analysis format (multi-plate reader) is used for standard clinical laboratory diagnostic tests (e.g., HIV, Lyme disease antigen and antibody tests). The key components needed to measure Cys and CysS from WF samples are a commercially available validated assay kit (Abcam; ab211099).

### Acceptability of Candidate Biomarker

Augmenting clinical data with scientific data provide additional weight of evidence. Scientific understanding includes the biological rationale, understanding of the molecular mechanisms, and the link of the proposed biomarker to regulatory understanding of the scientific impact. The candidate biomarkers proposed in this study are acceptable for the following reasons:
The ratio of Cys/CysS has been proposed to serve as a biomarker of tissue oxidative stress in animals and humans.
In the context of chronic wounds such as DFUs, prolonged inflammation and infection both promote oxidative stress and lead to impaired wound healing.
Cysteine is an important amino acid in proteins that undergoes reversible oxidation/reduction under biologic conditions and has been shown to not only control protein function, but also serve as a biomarker of oxidative damage.
Low cysteine has been linked to diabetes and other diseases like stroke, cancers, cardiovascular diseases etc.
Amino acid biomarkers are being studied in the context of various diseases, including diabetes, cancer, Alzheimer's disease among others.
Rigorous interrogation of 578 metabolites from 161 chronic wounds (including 76 diabetic wounds) identified Cys/CysS as candidate biomarker
Low molecular weight metabolites as biomarker candidates are already available or being confirmed for various human diseases.
Ratio of branched-chain amino acids (BCAAs) to aromatic amino acids, known as Fischer's ratio, is an established diagnostic marker used to monitor the progression of liver fibrosis and the effectiveness of drug treatments.

Cystine levels are of clinical importance in conditions such as cystinuria and juvenile nephropathic cystinosis and therefore clinical diagnostic tests for CysS are available. Enzyme assay platforms for qualitative, semi-quantitative or quantitative determination are routinely used in clinical diagnostics for the detection of various molecules/metabolites from human bodily fluids (e.g., bacterial and viral antigens or antibodies in serum/plasma samples). Therefore, the detection method proposed for phase II CT is feasible for studying the candidate biomarker utilizing existing clinical diagnostic test setup in clinical chemistry laboratories. The assay kit proposed can be used at Point-of-Care.

In accordance with embodiment 1, a method of treating a chronic wound in a patient is provided, wherein the method comprises identifying a patient as having a chronic wound by obtaining a wound fluid sample from a patient's wound; and identifying an altered metabolite profile, based on metabolites detected in said wound fluid sample, that is associated with chronic wounds; and treating said patient identified as having a chronic wound with advanced wound healing therapy.

In accordance with embodiment 2 the method of embodiment 1 is provided wherein the metabolites are detected and analyzed through the use of mas spectroscopy.

In accordance with embodiment 3 the method of embodiment 1 or 2 is provided wherein the altered metabolite profile comprises a difference in the concentration of a thiol bearing metabolite.

In accordance with embodiment 4 the method of any one of embodiments 1-3 is provided wherein the altered metabolite profile comprises the relative concentration of cysteine and cystine.

In accordance with embodiment 5 the method of any one of embodiments 1-4 is provided wherein a cysteine to cystine ratio of less than 3.4 identifies a chronic wound.

In accordance with embodiment 6 the method of any one of embodiments 1-5 is provided wherein the altered metabolite profile comprises an alteration in the concentration (either an increase or decrease relative to concentration present in healing wound fluids) of one or more metabolites selected from the group consisting of glycylglutamate, cysteine, pro-hydroxy-pro, 5-hydroxylysine, prolylserine, cystine, taurine, cysteine s-sulfate, oleoyl ethanolamide, pregnenediol disulfate (C₂₁H₃₄O₈S₂), cytidine 5′-diphosphocholine, 1-(1-enyl-oleoyl)-GPE (P-18:1), 3-methoxycatechol sulfate (1), cytidine 3′-monophosphate (3′-CMP), phosphoethanolamine, gamma-glutamylphenylalanine, choline phosphate, methionylvaline, O-sulfo-L-tyrosine, guanosine 5′-monophosphate (5′-GMP), N6-carbamoylthreonyladenosine, cytidine, 3-hydroxybutyrylcarnitine, valyltyrosine, and methionylalanine relative to the corresponding metabolic profile detected in would fluid from a healing wound.

In accordance with embodiment 7 the method of any one of embodiments 1-6 is provided wherein the altered metabolite profile comprises a change in the ratio of two metabolites detected in the wound fluid of a patient (relative to the ratio of the same two metabolites detected in healing would fluids), wherein the ratio is between a first metabolite selected from cysteine, taurine, cytidine 5′-diphosphocholine, cytidine 3′-monophosphate (3′-CMP), phosphoethanolamine, choline phosphate, methionylvaline, guanosine 5′-monophosphate (5′-GMP), 3-hydroxybutyrylcarnitine, valyltyrosine, and methionylalanine and a second metabolite selected from the group consisting of glycylglutamate, pro-hydroxy-pro, 5-hydroxylysine, prolylserine, cystine, cysteine s-sulfate, oleoyl ethanolamide, pregnenediol disulfate (C₂₁H₃₄O₈S₂), 1-(1-enyl-oleoyl)-GPE (P-18:1)*, 3-methoxycatechol sulfate (1), gamma-glutamylphenylalanine, O-sulfo-L-tyrosine, N6-carbamoylthreonyladenosine, and cytidine.

In accordance with embodiment 8 the method of any one of embodiments 1-7 is provided wherein the altered metabolite profile comprises an elevated level of one or more metabolites, relative to levels detected in healing wounds, wherein said metabolites are selected from glycylglutamate, pro-hydroxy-pro, 5-hydroxylysine, prolylserine, cystine, cysteine s-sulfate, oleoyl ethanolamide, pregnenediol disulfate (C₂₁H₃₄O₈S₂), 1-(1-enyl-oleoyl)-GPE (P-18:1), 3-methoxycatechol sulfate (1), gamma-glutamylphenylalanine, O-sulfo-L-tyrosine, N6-carbamoylthreonyladenosine, and cytidine.

In accordance with embodiment 9 the method of any one of embodiments 1-8 is provided wherein the altered metabolite profile comprises a decreased level of one or more metabolites, relative to levels detected in healing wounds, wherein said metabolites are selected from cysteine, taurine, cytidine 5′-diphosphocholine, cytidine 3′-monophosphate (3′-CMP), phosphoethanolamine, choline phosphate, methionylvaline, guanosine 5′-monophosphate (5′-GMP), 3-hydroxybutyrylcarnitine, valyltyrosine, and methionylalanine.

In accordance with embodiment 10 the method of any one of embodiments 1-9 is provided wherein the altered metabolite profile comprises an elevated level of one or more metabolites, relative to levels detected in healing wounds, wherein said metabolites are selected from glycylglutamate, 5-hydroxylysine, pro-hydroxy-proline, 2-hydroxyhippu, beta-hydroxyls, and naproxen.

In accordance with embodiment 11 the method of any one of embodiments 1-10 is provided wherein the altered metabolite profile comprises a difference in the concentration of 5-hydroxyproline, c-glycosyltryptophan, cysteinylglycine, guanidinoacetate, kynurenine, N(1)-acetylspermine, N1, N12-diacetylspermine, or taurine in wound fluid recovered from said patient relative to concentrations found in healing wound fluids.

In accordance with embodiment 12 the method of any one of embodiments 1-11 is provided wherein the altered metabolite profile comprises a difference in the concentration of one or more of the following: 3-phosphoglycerate, fructose 1,6-diphosphate/glucose 1,6-diphosphate/myo-inositol diphosphates, glucose, maltose, phosphoenolpyruvate, ascorbate, 5-methyluridine, cytidine 3′-monophosphate, cytidine 5′-monophosphate, guanosine 3′-monophosphate, guanosine 5′-monophosphate, inosine, uridine 2′-monophosphate, ergothioneine, levulinate, methyl-4-hydroxybenzoate, or 3-phosphoglycerate, in wound fluid recovered from said patient relative to concentrations found in healing wound fluids.

In accordance with embodiment 13 the method of any one of embodiments 1-12 is provided wherein the altered metabolite profile comprises a difference in the concentration of: glycylglutamate and/or isoleucyltyrosine in wound fluid recovered from said patient relative to concentrations found in healing wound fluids.

In accordance with embodiment 14 the method of any one of embodiments 1-13 is provided wherein said advanced therapy comprising administering therapy selected from the group consisting of wound debridement, negative pressure therapies, electrical stimulation, compression therapy and surgical procedures to alleviate ischemia.

In accordance with embodiment 15 the method of any one of embodiments 1-14 is provided wherein the wound is an ulcer, infectious wound, ischemic wound, surgical wound, or wounds from radiation.

In accordance with embodiment 16 the method of any one of embodiments 1-15 is provided the wound is in a diabetic patient.

In accordance with embodiment 17 the method of any one of embodiments 1-16 is provided wherein the wound is a diabetic foot ulcer (DFU).

In accordance with embodiment 18 a method of treating chronic wounds in a subject is provided wherein the method comprises:
a) receiving an identification of the subject as having a chronic wound, wherein
   the chronic wound has been identified by a detection of an altered metabolite of any one of embodiments 1-13, optionally including a low cysteine to cystine ratio of less than 3.4, in would fluid recovered from the subject's wound; and
b) administering advanced wound healing therapy to the subject identified as having a chronic wound.

### EXAMPLE 1

Pipe line reflecting the development of candidate metabolite biomarker for DFU non-healing.

The proposed work seeks to establish Cysteine Redox as a biomarker of non-healing or open wound, with the study named CREDO. The R61 preparatory phase is named 2CREDO (towards CREDO), and the R33 phase is referred to as CREDO. The discovery phase included 161 wound fluids from healing (H) and non-healing (NH) chronic wounds of which 77 were diabetic ulcers. 578 metabolites (amino acids, carbohydrates, vitamins, etc) were short listed from metabolomics analysis as unique between H and NH wounds. Statistical analysis (FIGS. 1-4B) short listed one candidate biomarker (cysteine/cystine (Cys/CysS) as a potential candidate. A point-of-care kit-based assay verified that Cys/CysS is significantly associated with non-healing DFU (FIG. 5).

The proposed 2CREDO (R61) studies will rest on this firm foundation to collect data from a broader DFC demographics. Following the Discovery Phase that typically includes internal validation, candidate biomarkers will be adapted to clinically applicable assay platforms and subjected to two types of validation: i) analytical validation: we will determine the accuracy and reliability of the test to measure the analytes of interest in the patient specimen; and ii) clinical validation: we will assess the robustness and reliability of assay. The results will be correlated with DFU non-healing. Sample collection: (i) we will obtain adequate amounts of fluid (undiluted volumes: Negative Pressure Wound Therapy (NPWT): 2-5 ml; disk filter: 250 μl, filter disk samples are ˜10× more concentrated that NPWT dressing); samples will be diluted prior to assay; (ii) optimize sample collection in weak acid such that both Cys and CysS are maximally recovered and their redox state are stabilized; (iii) properly preserve samples so as arrest Cys/CysS ratio during storage; and (iv) determine appropriate controls.

Metabolomics analysis was performed using an ultra-performance liquid chromatography and a Q-Exactive high resolution/accurate mass spectrometer interfaced with a heated electrospray ionization source and Orbitrap mass analyzer operated at 35,000 mass resolution. The analytical platform used has been heavily published by several groups. N=161 (n=76 diabetic ulcers) chronic wound fluids were included in the unbiased screening analysis. Sample extracts were analyzed under four different conditions for hydrophilic, hydrophobic, basic negative ions and negative ions. The MS analysis alternated between MS and data-dependent MSn scans using dynamic exclusion. Scan range varied slighted between methods but covered 70-1000 m/z. Raw data was extracted peak-identified and QC processed independent of the investigators. Compounds were identified by comparison to library entries of purified standards or recurrent unknown entities. The library contains the retention time/index (RI), mass to charge ratio (m/z), and chromatographic data (including MS/MS spectral data) on all molecules. Furthermore, biochemical identifications were based on three criteria: retention index within a narrow RI window of the proposed identification, accurate mass match to the library +/−10 ppm, and the MS/MS forward and reverse scores between the experimental data and authentic standards. The use of all three data points helped to reliably differentiate metabolites. Instrument variability was determined by calculating the median relative standard deviation (RSD) for the internal standards that were added to each sample prior to injection into the mass spectrometers. Overall process variability was determined by calculating the median RSD for all endogenous metabolites present in 100% of the technical replicates of pooled samples.

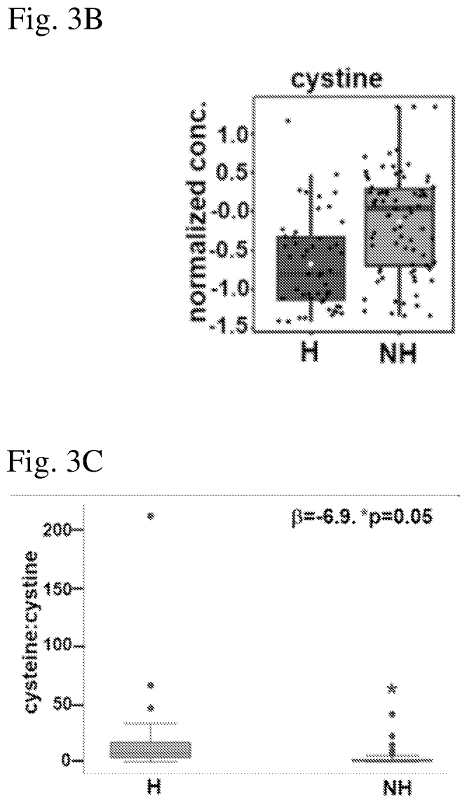

From these analyses, ↓Cys/CysS ratio was found to be significantly associated with non-healing (FIGS. 3A-5). Preliminary data from prospective DFU WF (N=24) analyses determined that ↓Cys/CysS was significantly associated with non-healing (FIG. 5). Among non-healers (NH) the Cys to CysS ratio was significantly downregulated two-fold (Coeff=−2.07) compared to H (p=0.036). ROC analysis of these biomarkers with wound healing outcome among all diabetic ulcers revealed that the area under the curve (AUC) for cysteine: cystine was 0.81. According to Youden's index, the recommended cut-off value to maximize sensitivity and specificity of DFU healing outcome prediction was a WF ratio of 3.43 (78% sensitivity, 75% specificity) (See Table 1).

TABLE 1

| | | | correctly | |
|---|---|---|---|---|
| cutpoint | sensitivity | specificity | classified | Youden |
| (>=.0046 | 1.0 | 0.03 | 0.48 | 0.03 |
| (>=.4672) | 0.96 | 0.24 | 0.57 | 0.21 |
| (>=.5436) | 0.96 | 0.27 | 0.59 | 0.24 |
| (>=.6140) | 0.96 | 0.30 | 0.61 | 0.27 |
| (>=.7568) | 0.96 | 0.33 | 0.62 | 0.30 |
| (>=.8278) | 0.96 | 0.39 | 0.66 | 0.36 |
| (>=.91051) | 0.96 | 0.42 | 0.67 | 0.39 |
| (>=1.021) | 0.93 | 0.45 | 0.67 | 0.38 |
| (>=1.1071) | 0.93 | 0.52 | 0.70 | 0.44 |
| (>=1.21884) | 0.93 | 0.55 | 0.72 | 0.47 |
| (>=1.455) | 0.89 | 0.61 | 0.74 | 0.50 |
| (>=2.255) | 0.82 | 0.64 | 0.72 | 0.46 |
| (>=2.703) | 0.79 | 0.64 | 0.70 | 0.42 |
| (>=2.811) | 0.79 | 0.67 | 0.72 | 0.45 |
| (>=2.815) | 0.79 | 0.70 | 0.74 | 0.48 |
| (>=2.867) | 0.79 | 0.73 | 0.75 | 0.51 |
| (>=3.426) | 0.79 | 0.76 | 0.77 | 0.54 |
| (>=3.538) | 0.75 | 0.76 | 0.75 | 0.51 |
| (>=3.615) | 0.71 | 0.76 | 0.74 | 0.47 |
| (>=4.002) | 0.71 | 0.79 | 0.75 | 0.50 |

Table 1: ROC analysis of Cys/CysS in wound fluids. The cutoff based on Youden Index for healing rate was >/=3.43 which indicates that Cys/CysS<3.43 will serve as an indicator of non-healing trajectory. Youden Index gives the performance of the biomarker in predicting wound healing at the given cutoff. Higher the number, the more perfect the prediction will be. For brevity, only select values are shown in Table 1.

To ensure the rigor, high sensitivity, precision, and accuracy of the proposed biomarkers, DFU WF samples were tested using a targeted fluorimetric assay. This assay (Abcam, MA, ab211099120-122) is intended to be the Point of Care (PoC) assay. The assay was able to detect Cys and CysS in all DFU samples. To further validate the PoC assay, the gold standard for thiol detection high performance liquid chromatography electrochemical detection (HPLC-EC) was employed. PoC Assay: Standards (10 μl) or WF (10 μl) was pipetted into two wells (one well for free cysteine and one well for total cysteine; total−free cysteine=cystine or CysS) of the microtiter plate. Two consecutive steps of incubation at 37° C. were performed for 30 mins (reaction mix 1) and 5 mins (reaction mix 2). Following this, 5 μL of Cys probe was added and fluorescence was measured immediately at Ex/Em=365/450 nm in kinetic mode for 30 minutes at room temperature. The difference between the total cysteine and the free cysteine corresponded to the amount of cystine in the sample (that was chemically reduced to cysteine in the reaction). The results indicated that

Cys to CysS ratio was significantly lower in non-healing (NH) compared to healing DFU WF (FIG. 5). These results corroborated the findings of our metabolomics analyses (FIGS. 1-4B).

Studies proposed in the 2CREDO (R61) and CREDO (R3) phases will establish the predictive power and determine CoU the biomarker based on multi-center data in a rigorous consortium (DFC) setting.

### Rigor, Transparency and Reproducibility

Prospectively defined exclusion/inclusion criteria for subject enrollment will be used to generate robust results that are widely applicable to most DFU patients. All analyses will be performed in a double-blinded manner i.e., DFU healing outcome (masked from laboratory study team) will be handed over to an independent biostatistician for statistical analyses. Appropriate statistical methods, prospective sample size estimation, replicates and standards (reference reagents or data standards) will be employed. Complimentary and redundant approaches will be used in experimental designs to ensure rigor. We recognize that biases such as: a. analytical errors, b. subject non-compliance with study parameter and c. equipment function may affect the clinical interpretation of the accuracy of the WF Cys/CysS as a biomarker for DFU non-healing. To obviate these biases, the following countermeasures are proposed: i. An SOP based on our pilot studies will be followed rigorously that includes a minimum of 3 technical replicates for Cys/CysS analysis. Structured training will be provided to all staff performing the assays. ii. Every effort will be made to ensure that subjects complete the study (including a nominal subject compensation for each study visit). Biological variables: Sex: For the study, both adult males and females will be included in the study. Gender: Binary gender as well as gender minorities including transgender, non-binary or gender nonconforming subjects will be enrolled.

A robust ultrahigh performance liquid chromatography-tandem mass spectroscopy (gold standard) platform combined with blinded bioinformatics analysis performed by an independent facility was adopted. This analysis was able to screen 578 metabolites from 161 patients. The metabolomic approach afforded unbiased query of a large number of metabolites analyzed in a single run. Of all these metabolites, ↓Cys/CysS emerged as a robust predictor of non-healing.

The amino acid Cysteine (Cys) is known to be a rate-limiting precursor for protein synthesis and function. The oxidation product of Cys is cystine (CysS). Thus, Cys-CysS forms a redox pair. The redox state of Cys, Cys/CysS, is known to be a marker for oxidative stress. Two well-known factors characteristic of DFU are known to exacerbate oxidative stress: (i) chronic inflammation, and (ii) infection. Both conditions produce copious amounts of reactive oxygen/nitrogen species (RONS) that may oxidize a nucleophilic thiol like Cys thus causing ↓Cys/CysS. A prospective preliminary study on DFU patients (N=24) show that WF ↓Cys/CysS predicts non-healing. A point-of-care microtiter-plate (standard hospital assay platform) based test was used.

In further clinical studies we will seeks to collect WF samples and wound healing outcomes from DFU patients from diverse demographics consortium-wide to confirm the clinical validity of WF↓Cys/CysS ratio as a prognostic biomarker for non-healing DFU. The studies will be conducted with DFC as Ancillary Study (DFC approved) taking advantage of the Biorepository Program. Any DFU patient consented by the DFC will be sought for WF collection. The proposed study will not compete with any ongoing/existing DFC study protocols because the sample collected will use a standard of care (NPWT) dressing or filter disk. Since NPWT dressings are removed and discarded during scheduled dressing changes, they will be collected for study as available. It has been statistically determined that a total of N=98 (n=58 for training; n=40 for independent testing) samples will be required for the this study. The independent testing dataset will evaluate internal validation of the models with the biomarker in predicting wound healing outcomes. In the interest of broad demographics, as many samples of N=98 as can be obtained from DFC in one year (2CREDO duration) will be included. The balance, if any, will be made up by currently prospectively collected DFU WF samples on site (currently 62 new samples collected for this study and banked; these new samples are in addition to the samples from which all preliminary data are shown). We propose 2CREDO as a 12-weeks prospective observational clinical study where adult patients with DFUs; open >30 d, the CMS definition for chronic wounds, already enrolled in DFC biorepository protocol will be the study subjects. One WF sample will be collected for analysis during first visit or within 4 weeks of first visit. Wound closure data will be collected over 12 weeks or when wound closes (whichever is later). Primary outcomes: Rate of wound closure at 12 weeks from first visit to wound clinic; Secondary outcomes: a. complete wound healing at 12 weeks, b. rate of wound closure at 4 weeks, c. amputation rate. Patient reported outcomes: a. general HRQOL (SF 36) 125; b. wound-specific HRQOL (Cardiff Wound Impact Schedule) 126; and c. pain symptoms (Visual Analog Scale for Pain).

### Biospecimen Collection

NPWT Dressings: NPWT dressings will be collected at the time of study. The tubing and plastic base of tubing will be removed with scissors. The dressing will be placed in a sealing (e.g. Ziploc) bag with a biohazard label containing a stabilizing solution. For those patients not on NPWT as part of SoC, the filter disc approach will be adopted. Wound fluid using Whatman paper discs: This approach is applicable for exudative wounds as reported. Briefly, sterile (10 mm) Whatman paper discs up to six will be placed on open wounds for 5 minutes or earlier if the paper gets saturated. WF saturated filter discs will be placed in a plastic container containing stabilizing solution. Collected either way, specimens are held on ice (<15 min) until storage in a −80° C. freezer or liquid N₂ (whichever is available) within 0.5-1 h of collection. The specimen will be placed on ice and maintained on ice until storage in a −80° C. freezer or liquid N₂ (whichever is available) within 0.5-1 h of collection. One set of samples will be measured using the PoC assay kit and another set will be shipped overnight to the BAU on dry ice following standard shipping protocols. Note that each PoC assay requires only few microliters of the sample in a microtiter plate format. Cys/CysS PoC Assay.

As discussed above, in one embodiment a high throughput multi-plate fluorimeter (Abcam, MA, ab211099) based assay will be used to detect Cys/CysS. The principle of the assay is based on the cleavage of thiol group of reduced cysteine producing a fluorophore (Ex/Em =365/450 nm) with a stable signal, which is directly proportional to the amount of total cysteine in the sample. Agreement of data from PoC kit based assay will be test using HPLC-EC. Electrochemical (EC) detectors have higher specificity and sensitivity (˜100×) towards compounds than standard UV-based detection. On a HPLC column, Cys and CysS separate clearly. Retention times: Cys=3.5 mins, >500 mV; CysS=3.0 mins, >900 mV; Column (C18 RP): 150 mm, 5 μm, Hichrom). Separation is achieved using solvents over 25 min at a flow rate of 0.6 ml/min with a mixture (50:50) of mobile phase A (Water) and mobile phase

B (0.05% trifluoroacetic acid in water (98.5%) and methanol (1.5%)). 120-122 The electrochemical detector functioned in DC mode where the detector potential was set between 400 mV-1000 mV with a 50-100 mV increments. Data normalization.

The data used for assessing the biomarker candidate is represented as a ratio of Cys over CysS. This method intrinsically normalizes the data by adjusting for the differences in the denominator to look at how the numerator affects the outcome. Therefore, the ratio would be more specific to individual patient's data in our study population.

### Data Analysis and Statistics

We will utilize data primarily from prospective samples (collected by DFC in a year; and a balance of banked samples data to achieve N=98 in one year) for statistical analysis. Baseline wound fluid from DFUs will be obtained at the time of the patient's first visit. The wound fluid will be then be evaluated for Cys/CysS ratio. Normality of these biomarkers will be tested using Shapiro-Wilk test and appropriate log-transformation will be done as needed to satisfy the model assumptions. Using the prospective data, henceforth referred to as training data, we will use the values of each of these biomarkers to predict the probability of DFU healing status using multivariable logistic regression models (EQ.1) after adjusting for the confounding effects of age, gender, and comorbidities. In addition to these variables, set of other relevant confounding variables, selected by various association analysis techniques, published literature, and experiences of wound experts, will be obtained to represent ulcer history, medical history, concomitant medication, and therapy use, social factors, focused physical examination, and quality of life assessment to be considered for multivariable model.

Penalized logistic regression or a random forest for classification will be used to select the appropriate confounders for the multivariable logistic regression to predict DFU wound non-healing. Adjusted odds ratios with 95% confidence intervals and p-values will be used to quantify the effect of the proposed biomarker on wound healing. Marginal effects with marginal plots will be used to visualize the probability of predicting wound healing based on different values of the biomarker in the training dataset. The discriminative performance—ability to distinguish between healed vs not-healed wounds-of the models will be evaluated using Receiver Operating Characteristics (ROC) Curve or concordance (C)-statistics. Various thresholds of sensitivity and specificity of each biomarker will be examined using Youden Index and the optimal value of these biomarkers with maximum Youden Index will be reported as a cutoff for these biomarkers to predict wound healing with optimal sensitivity and specificity. In addition to assessing the predictive performance using discriminative ability, calibration and overall accuracy will also be evaluated. Calibration will be assessed by comparing the predicted probabilities with observed probabilities of DFU healing and will be quantified using “calibration slope”. A well calibrated model will have the slope equal to or close to 1 while the model with extreme predictions will have a slope less than 1. For accuracy, Brier score, which is an aggregate measure of disagreement (the average squared error difference) between the observed and predicted outcomes will be reported. Both adjusted and unadjusted models for each biomarker in the training data will be examined with 95% confidence intervals for the effect sizes. Internal validation will be done to examine the potential usefulness of Cys/CysS as a biomarker based on sensitivity and specificity of these biomarkers in distinguishing between healing vs non-healing wound in clinical practice. Banked data coming from the sample population and clinical setting in Indiana, independent from the training dataset, will be used as an independent testing dataset to evaluate internal validation of the models with the biomarkers in predicting wound healing outcomes. Multiple site investigation will be performed in R33 phase to evaluate the general external validity (generalizability) of the biomarkers in predicting wound healing. However, this split-sample like approach may underestimate the full model performance since 40% of the banked sample data is not used in the model building step. Thus, to overcome this, we will use 5-fold cross-validation method in the full dataset (N=98; assuming prospectively collected by DFC n=58 with 10% attrition and banked at IU, n=40) where all the observations will be split into five random subsets of dataset. Leaving one-set out (as a validation subset), the model will be developed in the remaining four sets and will then be cross-validated in the left-out validation subset. We will then repeat the process until all subsets will be consecutively treated as the validation subset. Next the cross-validation estimate of the prediction error for the proposed model will be estimated. All prediction model reporting will be done based on the Transparent Reporting of a multivariable prediction model for Individual Prognosis or Diagnosis (TRIPOD).

The obvious benefit derived from these measurements is an ‘early warning system’ that alerts the attending physician of SoC failure in DFU management.

Based on the data obtained from 2CREDO (R61) pilot studies, a complete clinical protocol will be drafted, per DFC template, for a multi-center clinical study of diabetic foot ulcer patients to perform detailed validation of wound fluid-based Cys/CysS ratio as biomarkers to predict diabetic foot ulcer healing via DFC.

The following key areas will be considered while developing the clinical study protocol: i) title, ii) description, iii) primary and secondary objectives, iv) study endpoints, v) study population and eligibility criteria, vi) participating study sites, vii) study duration, ix) study activities, assessments and schedule x) Risk Benefit assessments, xi) safety and assessments, xii) AEs and SAEs, xiii) procedures, xiv) statistical considerations including sample size estimations. Informed consent form and HIPAA authorization. An Informed Consent Form (ICF) and HIPAA authorization will be developed. The informed consent will be obtained in accordance with the Declaration of Helsinki, ICH GCP, US Code of Federal Regulations for Protection of Human Participants (21 CFR 50.25[a,b], CFR 50.27, and CFR Part 56, Subpart A), the Health Insurance Portability and Accountability Act (HIPAA, if applicable), and local regulations. Study Design. In consultation with the DCC of DFC, we will develop protocol for a multicenter observational study of DFU patients aimed to determine clinical validity of WF Cys/CysS as an early biomarker for non-healing. Patients will be eligible to participate in the study if they have an active DFU and comply with certain inclusion and exclusion criteria. The collections will consist of WF obtained from DFU patients consented under the approved DFC protocols.

At the completion of the 2CREDO R61 phase, the following outcomes are anticipated: •collection, processing and analysis of N=98 DFU samples •successful analytical validation of detection assay •early proof-of-concept of correlation between ↓Cys/CysS ratio and non-healing DFU •development of standard operating protocols (SOP) for systematic study from fluid collection to final analysis

### CREDO (R33) PHASE

Aim 3: Conduct a multi-center clinical study of diabetic foot ulcer patients to perform detailed validation of wound fluid-based Cys/CysS ratio as a biomarker to predict diabetic foot ulcer healing. SOPs developed at the end of 2CREDO will be critically examined by the DFC steering and ancillary studies committees to launch the independent confirmation study (n=172). An anticipated design outline is proposed based on current experience within the DFC. Note: PI is a member of the DFC steering and ancillary studies committee.

### Overall Study Design

The proposed ancillary study will leverage the multi-center clinical research infrastructure and wisdom of the DFC. Any eligible patient with a DFU open wound will be consented and enrolled at visit 1 (enrollment) during which time a WF sample will be collected. Wound closure data will be collected over 12 weeks or when wound closes (whichever is later). The study is not expected to compete/interfere with any ongoing protocols.

### Study Inclusion/Exclusion Criteria

Inclusion Criteria:
adult aged 18 years or older •foot ulcer of diabetic etiology with an ulcer duration of >4 weeks.
patients willing to comply with all study procedures and have signed and dated informed consent form.

Exclusion Criteria:
individuals who are deemed unable to understand the procedures, risks and benefits of the study, ie. Informed consent.
individuals who are therapeutically immuno-compromised. •candidates for inclusion into the study will not include individuals as defined in 45CFR46 SubpartsB, C and D, nor from any other population which may be considered vulnerable.

Study Population: Potential patients will be identified via patient chart review by pre-screening at clinics. The approved research staff will work around wound clinic schedules for patients diagnosed with a diabetic foot ulcer. A patient that is consented by DFC will be sought for WF collection (NPWT dressing if available or filter disk. Study staff will follow wound closure of these patients per regular schedule.

Aim 4: Initiate discussions with FDA to establish Cys/CysS as a clinical biomarker

The process with FDA will begin by directing pre-submission or general inquiry or meeting requests with the help of regulatory experts (Pearl Pathways; see LoS)). Biomarker qualification is a process involving three stages that provide increasing levels of detail for the development of a biomarker for its proposed context of use (COU). FDA has detail instructions available on their site for complete submissions to the CDER Biomarker Qualification Program. Three major steps for Biomarker Qualification involves: i) Letter of Intent (LOI); ii) Qualification Plan (QP); iii) Full Qualification Package (FQP). Per FDA guidance, the following framework will be followed for biomarker qualification:

Biomarker Information and Interpretation. High level descriptions of the biomarker including: a. Biomarker name: abbreviated short name for biomarker, or names if multiple, AND identify each biomarker type (molecular, histologic, radiographic, or physiologic characteristics according to BEST Glossary), b. Analytical methods: Name and brief description of the analytical methods, c. Measurement units and limit(s) of detection, d. Biomarker interpretation and utility. A brief description of how the raw biomarker measurement will be used/applied. Clinical Interpretive Criteria will include description of cut-off values, cut-points/thresholds, boundaries/limits.
ii) Context of Use Statement. By the end of CREDO phase, a defined COU statement will be developed following FDA guidelines.

Analytical Considerations. A general description of what aspect of the biomarker is being measured and by what method. A brief description of sample source, matrix (base material and any additives), stability and composition of biomarker.

Clinical Considerations. Details on the patient population and clinical validation process supporting the biological and clinical relevance of the biomarker will be clearly defined based on 2CREDO and CREDO phases with input from the DFC.

1. A method of treating a chronic wound in a patient, said method comprising
identifying a patient as having a chronic wound by
obtaining a wound fluid sample from a patient's wound; and
identifying an altered metabolite profile, based on metabolites detected in said wound fluid sample, that is associated with chronic wounds; and
treating said patient identified as having a chronic wound with advanced wound healing therapy.

2. The method of claim 1 wherein said metabolites of the wound fluid are analyzed using mass spectrometry to identify said altered metabolite profile of the wound fluid.

3. The method of claim 1 wherein the altered metabolite profile comprises a difference in the concentration of a thiol bearing metabolite.

4. The method of claim 1 wherein the altered metabolite profile comprises the relative concentration of cysteine and cystine.

5. The method of claim 4, wherein a cysteine to cystine ratio of less than 3.4 identifies a chronic wound.

6. The method of claim 1 wherein the altered metabolite profile comprises a detected alteration in the concentration of one or more metabolites selected from the group consisting of glycylglutamate, cysteine, pro-hydroxy-pro, 5-hydroxylysine, prolylserine, cystine, taurine, cysteine s-sulfate, oleoyl ethanolamide, pregnenediol disulfate (C₂₁H₃₄O₈S₂), cytidine 5′-diphosphocholine, 1-(1-enyl-oleoyl)-GPE (P-18:1), 3-methoxycatechol sulfate (1), cytidine 3′-monophosphate (3′-CMP), phosphoethanolamine, gamma-glutamylphenylalanine, choline phosphate, methionylvaline, O-sulfo-L-tyrosine, guanosine 5′-monophosphate (5′-GMP), N6-carbamoylthreonyladenosine, cytidine, 3-hydroxybutyrylcarnitine, valyltyrosine, and methionylalanine, relative to the corresponding metabolic profile detected in would fluid from a healing wound.

7. The method of claim 6 wherein the altered metabolite profile comprises a change in the ratio of two metabolites detected in the wound fluid, wherein the ratio is between a first metabolite selected from cysteine, taurine, cytidine 5′-diphosphocholine, cytidine 3′-monophosphate (3′-CMP), phosphoethanolamine, choline phosphate, methionylvaline, guanosine 5′-monophosphate (5′-GMP), 3-hydroxybutyrylcarnitine, valyltyrosine, and methionylalanine and a second metabolite selected from the group consisting of glycylglutamate, pro-hydroxy-pro, 5-hydroxylysine, prolylserine, cystine, cysteine s-sulfate, oleoyl ethanolamide, pregnenediol disulfate (C₂₁H₃₄O₈S₂), 1-(1-enyl-oleoyl)-GPE (P-18:1)*, 3-methoxycatechol sulfate (1), gamma-glutamylphenylalanine, O-sulfo-L-tyrosine, N6-carbamoylthreonyladenosine, and cytidine.

8. The method of claim 6 wherein the altered metabolite profile comprises an elevated level of one or more metabolites, relative to levels detected in healing wounds, wherein said metabolites are selected from glycylglutamate, pro-hydroxy-pro, 5-hydroxylysine, prolylserine, cystine, cysteine s-sulfate, oleoyl ethanolamide, pregnenediol disulfate (C₂₁H₃₄O₈S₂), 1-(1-enyl-oleoyl)-GPE (P-18:1), 3-methoxycatechol sulfate (1), gamma-glutamylphenylalanine, O-sulfo-L-tyrosine, N6-carbamoylthreonyladenosine, and cytidine.

9. The method of claim 6 wherein the altered metabolite profile comprises a decreased level of one or more metabolites, relative to levels detected in healing wounds, wherein said metabolites are selected from cysteine, taurine, cytidine 5′-diphosphocholine, cytidine 3′-monophosphate (3′-CMP), phosphoethanolamine, choline phosphate, methionylvaline, guanosine 5′-monophosphate (5′-GMP), 3-hydroxybutyrylcarnitine, valyltyrosine, and methionylalanine.

10. The method of claim 8 wherein the altered metabolite profile comprises an elevated level of one or more metabolites, relative to levels detected in healing wounds, wherein said metabolites are selected from glycylglutamate, 5-hydroxylysine, pro-hydroxy-proline, 2-hydroxyhippu, beta-hydroxyls, and naproxen.

11. The method of claim 1 wherein the altered metabolite profile comprises a difference in the concentration of 5-hydroxyproline, c-glycosyltryptophan, cysteinylglycine, guanidinoacetate, kynurenine, N(1)-acetylspermine, N1, N12-diacetylspermine, or taurine in wound fluid recovered from said patient relative to concentrations found in healing wound fluids.

12. The method of claim 1 wherein the altered metabolite profile comprises a difference in the concentration of one or more of the following: 3-phosphoglycerate, fructose 1,6-diphosphate/glucose 1,6-diphosphate/myo-inositol diphosphates, glucose, maltose, phosphoenolpyruvate, ascorbate, 5-methyluridine, cytidine 3′-monophosphate, cytidine 5′-monophosphate, guanosine 3′-monophosphate, guanosine 5′-monophosphate, inosine, uridine 2′-monophosphate, ergothioneine, levulinate, methyl-4-hydroxybenzoate, or 3-phosphoglycerate, in wound fluid recovered from said patient relative to concentrations found in healing wound fluids.

13. The method of claim 1 wherein the altered metabolite profile comprises a difference in the concentration of: glycylglutamate and/or isoleucyltyrosine in wound fluid recovered from said patient relative to concentrations found in healing wound fluids.

14. The method of claim 1 wherein said advanced therapy comprising administering therapy selected from the group consisting of wound debridement, negative pressure therapies, electrical stimulation, compression therapy and surgical procedures to alleviate ischemia.

15. The method of claim 14 wherein the wound is an ulcer, infectious wound, ischemic wound, surgical wound, or wound from radiation.

16. The method of claim 15 wherein the wound is in a diabetic patient.

17. The method of claim 16 wherein the wound is a diabetic foot ulcer (DFU).

18. A method of treating chronic wounds in a subject, said method comprising:
a) receiving an identification of the subject as having a chronic wound, wherein the chronic wound has been identified by a detection of a low cysteine to cystine ratio of less than 3.4 in would fluid recovered from the subject's wound; and
b) administering advanced wound healing therapy to the subject identified as having a chronic wound.

19. A kit for recovering wound fluids, said kit comprising:
a negative pressure wound therapy (NPWT) dressing or filter disks; and
a 1×PBS solution comprising monochloroacetic acid (MCA).
